(19) United States
(12) Patent Application Publication
(10) Pub. No.: US 2018/0168975 A1
(43) Pub. Date: Jun. 21, 2018

(54) USE OF N-ALKYLAMIDE FOR THE MANUFACTURE OF A COSMETIC ANTIPERSPIRANT COMPOSITION

(71) Applicant: ROBERTET S.A., Grasse (FR)

(72) Inventor: Anthony PEGARD, Grasse (FR)

(73) Assignee: ROBERTET S.A., Grasse (FR)

(21) Appl. No.: 15/579,781

(22) PCT Filed: Jul. 5, 2016
(86) PCT No.: PCT/FR2016/051693

§ 371 Date: Dec. 5, 2017

(30) Foreign Application Priority Data
Jul. 7, 2015 (FR) 15/56438

(51) Int. Cl. A61K 8/42; A61K 8/9789; A61Q 15/00

(52) U.S. Cl. CPC A61K 8/42; A61Q 15/00; A61K 8/9789

(57) ABSTRACT

The cosmetic use of at least one n-alkyl amide as an antiperspirant. N-alkyl amides are selected in particular from among the following molecules: spilanthol, trans-pellitorine, cis-pellitorine, 2Z,4Z- or 2Z,4E-decadienoic acid-N-isobutylamide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, rachilleamide, sarmentine, 2E- or 3E-decenoic acid-N-isobutylamide, 3E-nonenoic acid-N-isobutylamide, homospilanthol, 2E,6Z,8E-decatrienoic acid-N-([2R]-2-methylbutyl)amide, 2E- or 2Z-decen-4-oic acid-N-isobutylamide, sanshools including in particular hydroxy sanshool, α-sanshool, hydroxy-α sanshool, hydroxy-β sanshool, δ-sanshool, hydroxy-γ sanshool, hydroxy-γ isosanshool, dehydro-γ sanshool, γ-sanshool, bungeanool, isobungeanool, dihydrobungeanool, or tetrahydrobungeanool, or the combinations thereof.

Perspiration allows the body to maintain its temperature when it is hot, sweat ensuring the skin cooling.

A high, even excessive perspiration can be disturbing.

During perspiration phases, the sweat glands secrete sweat which contains essentially water, but also minerals and lactate.

In humans, there are two types of sweat glands that differ in their functions and in the composition of the sweat they excrete:
the «eccrine» sweat glands; and
the «apocrine» sweat glands.

The eccrine sweat glands are by far the most numerous and are located on almost the whole body but especially on the palms of the hands, on the soles of the feet and on the forehead. Each of them is a simple, tubular and spiral gland, whose end, the glomerulus, lies in the thickness of the dermis or in the subcutaneous tissue.

The sweat secreted by the eccrine sweat glands is composed of 99% of water and electrolytes, in particular sodium chloride, and of about 1% of organic compounds, especially lactic acid. Although the sweat secreted by the eccrine sweat glands does not smell in itself, it can, in some conditions of maceration, cause bacterial infections or irritation.

The apocrine sweat glands are found in particular under the armpit. They are larger than the eccrine glands and their excretory duct opens into a hair follicle.

In addition to the basic components identical to those of sweat excreted by the eccrine glands, the sweat excreted by the apocrine glands also contains organic molecules (lipids and proteins) including pheromones which, once transformed by cutaneous bacteria, cause the so-called «sweat» odors.

In order to prevent the appearance of these bad odors, deodorant compositions more conventionally designated by «deodorants» are in particular used. Deodorants can act in different ways:
by masking bad odors, for example thanks to perfume ingredients;
by absorbing sweat and limiting the diffusion of «odorant» molecules, for example with talc;
by limiting the production of sweat, for example with the botulinum toxin,
by acting directly on the bacteria that, by metabolizing constituents of the apocrine sweat, give off the odor attributed to perspiration.

The compositions aimed at reducing sweat production and preventing the appearance of bad odors comprise antiperspirant active ingredients.

Among the available antiperspirant active ingredients, there are aluminum salts, zirconium salts, and aluminum/zirconium salts, in order to reduce the production of sweat. However, the topical use of aluminum, zirconium, and aluminum/zirconium salts seem to pose health safety problems, as described in the publications concerning adverse effects associated with the use of antiperspirants. In addition, the acidity of aluminum, zirconium, and aluminum/zirconium salts make them potentially irritating to the skin. Antiperspirants based on aluminum, zirconium and aluminum/zirconium are also sensitive to metals, such as iron, which can destabilize the antiperspirant and cause the formation of strains on fabric.

Yet, it has now been found that compounds of the N-alkylamides family can quite surprisingly limit the production of sweat.

The present invention therefore relates to the use of at least one N-alkylamide for the preparation of an antiperspirant cosmetic composition.

The composition of the invention makes it possible to reduce the perspiration effectively, and thus the moisture associated with perspiration and bad odors, whether in the absence of an active aluminum-based antiperspirant or in combination with low amounts of an active aluminum and/or zirconium-based antiperspirant.

The N-alkymamides can be synthetic products or natural plant extracts. Among the N-alkylamides that can be used in the context of the invention there may be mentioned: spilanthol, trans-pellitorin; cis-pellitorin; 2Z,4Z- or 2Z,4E-decadienoic acid-N-isobutylamide; 2E,4E-decadienoic acid-N-([2S]-2-methylbutyl)amide; 2E,4E-decadienoic acid-N-([2R]-2-methylbutylamide); 2E,4Z-decadienoic acid-N-(2-methylbutyl)amide; achilleamide; sarmentine; 2E- or 3E-decenoic acid-N-isobutylamide; 3E-nonenoic acid-N-isobutylamide; homospilanthol; 2E,6Z,8E-decatrienoic acid-N-([2R]-2-methylbutyl)amide; 2E- or 2Z-decen-4-oic acid-N-isobutylamide; sanshools comprising in particular hydroxysanshool; α-sanshool; α-hydroxysanshool; β-hydroxysanshool; β-sanshool; γ-hydroxysanshool; γ-hydroxyisosanshool; γ-dehydrosanshool; γ-sanshool; bungeanool; isobungeanool; dihydrobungeanool; or tetrahydrobungeanool, or the combinations thereof.

Preferably, the cosmetic composition comprises at least two N-alkylamides. Indeed, the use of at least two N-alkylamides in one cosmetic composition according to the invention makes it possible to obtain a synergistic effect.

Preferably, the cosmetic composition comprises spilanthol and/or sanshools.

In one implementation of the invention, the source of spilanthol is an oleoresin extract of Acmella oleacerae or pure spilanthol and the source of sanshools is a supercritical CO₂ extract of Zanthoxylum piperitum.

By way of example, an oleoresin extract of Acmella oleacerae can comprise 32% spilanthol, a supercritical CO₂ extract of Zanthoxylum piperitum for example may comprise between 18 and 50% of sanshools.

According to another aspect of the invention, the amount of spilanthol in the composition is comprised between 0.05% and 10% by weight, advantageously 0.05% and 5% and more preferably between 0.05 and 1% and in particular 0.08% or 0.16% by weight of the composition. The amount in sanshools is comprised between 0.03% and 25% by weight, preferably between 0.04% and 5%, and preferentially, the amount of sanshools is comprised between 0.045 and 0.25% or between 0.09% and 0.125% by weight of the composition.

The cosmetic formulation may optionally comprise components that promote the N-alkylamides penetration. Examples of penetration enhancers comprise urea, polyoxyethylene (for example Brij-30 and laureth-4), 3-hydroxy-3,7,11-trimethyl-1,6,10-dodecatriene, cis-fatty acids (for example, oleic acid, palmitoleic acid), acetone, laurocapram, dimethylsulfoxide, 2-pyrrolidone, oleic alcohol, glycerol-3-stearate, propan-2-ol, ester of the isopropyl myristic acid, cholesterol, nerolidol, bisabolols and propylene glycol, as well as the derivatives thereof.

In another aspect of the invention, the penetration enhancer of the composition is the nerolidol.

The amount of penetration enhancer is comprised between 1 and 50% by weight of the cosmetic composition; preferably when the penetration enhancer is the nerolidol, the amount is comprised between 0.05 and 1% and preferentially of 1% by weight of the composition.

The N-alkylamides according to the invention are either integrated directly into the cosmetic composition or introduced into the cosmetic composition in the form a perfume, said perfume comprising the N-alkylamide(s) and optionally the penetration enhancer. In the case where the N-alkylamides are introduced in the form of a perfume, the perfume represents for example from 0.5 to 5% by weight of the cosmetic composition, preferably 1%.

According to another aspect, the invention also relates to a method for cosmetic treatment of the perspiration consisting in applying, locally, an effective amount of a composition based on at least one N-alkylamide. In a particular implementation, the applied composition comprises at least two alkylamides.

The invention also relates to a cosmetic composition comprising as active ingredient at least pure spilanthol or in the form of a Jambu oleoresin extract (Acmella oleracea) and/or sanshools in the form of supercritical CO₂ extract of Sichuan pepper (Zanthoxylum piperitum). This composition may also comprise a compound promoting the penetration of the active ingredients. In a particular implementation of the invention, the penetrating agent is the nerolidol.

In another implementation of the invention, the cosmetic composition comprises between 0.05% and 10%, advantageously 0.05% and 5%, and more preferably between 0.05 and 1% and in particular 0.08 or 0.16% of spilanthol by weight of the composition, and/or between 0.03 and 25%, advantageously from 0.04 to 5%, and in particular between 0.045 and 0.25% or between 0.09% and 0.125% of sanshools by weight of the composition and optionally 0.05 and 1%, and preferably 1% by weight of the nerolidol composition.

The cosmetic compositions according to the present invention can be formulated in any dosage form suitable for their administration. The compositions according to the present invention can thus be formulated in the form of cream, gel, lotion, milk, oil-in-water or water-in-oil emulsion, perfume, solution, ointment, spray, body oil, or stick.

According to another aspect, the invention also relates to the use of compositions described above as a deodorant.

The cosmetic compositions according to the present invention can be in the form of a cream or a gel in which at least one N-alkylamide, preferably two N-alkylamides, is mixed with the conventionally used excipients in the cosmetic field.

The cosmetic compositions according to the present invention can be in the form of gels in the appropriate excipients such as cellulose esters or other gelling agents, such as Carbopol, Sepinov (polyacrylate), guar gum, etc.

The cosmetic compositions according to the present invention can also be in the form of a lotion or a solution in which at least one N-alkylamide is in encapsulated form. The microspheres may for example consist of fat, agar and water. The N-alkylamides according to the present invention can be incorporated in liposome, glycosphere, cyclodextrin-type vectors, in chylomicrons, macro-, micro-, nanoparticles as well as macro-, micro- and nanocapsules and also be absorbed on powdery organic polymers, talcs, bentonites and other mineral supports.

These emulsions have a good stability and can be stored during the time required for use at temperatures comprised between 0 and 50° C. without sedimentation of the constituents or separation of the phases.

The cosmetic compositions according to the present invention can also contain additives or adjuvants commonly used in cosmetology, such as for example antimicrobial agents or perfumes but also extraction or synthetic lipids, gelling and viscosifying polymers, surfactants and emulsifiers, hydro- or liposoluble active ingredients, plant extracts, tissue extracts, marine extracts, synthetic active agents.

The cosmetic compositions according to the present invention can also comprise other complementary active ingredients selected for their action. When the compositions according to the present invention contain complementary active ingredients, these are generally present in the composition at a concentration high enough for them to carry out their activity.

The cosmetic compositions according to the present invention are preferably used daily and applied one or more times a day.

The present invention is illustrated in a non-limiting way by the following examples.

### EXAMPLES

### Example 1

The material used is the following:
cotton patches of a 7.5 cm×7.5 cm size

Raw materials used in perfumery:
Jambu Oleoresin (Acmella oleacerae) CAS No. 90131-24-1, containing 32% of spilanthol,
CO₂ extract of Sichuan pepper (Zanthoxylum piperitum) CAS No. 102242-62-6, containing 18% of alpha and beta-hydroxy sanshool,
1% of nerolidol CAS No 7212-44-4

Cotton patches allowing to collect the sweat produced during a reproducible effort are disposed under the armpits.

The perspiration produced during one hour of effort is then measured by weighing.

The difference between the (right) treated armpit and the (left) untreated armpit is measured, as well as the difference between right armpit treated with a placebo and right armpit treated with the active formula.

The effort is performed for one hour at a temperature of 20° C.

The experiments are performed in triplicate.

### Example 2

Application to the right armpit of 5 sprays (170 mg) of an 85% alcohol solution comprising either 0.5% of Jambu containing 32% of spilanthol (or 0.16% of pure spilanthol) and 1% of nerolidol is only 85% alcohol (control), and no application on the left armpit.

After the effort, at t+1 h, the weight of sweat collected on the right patches is measured:

Average of the Control (average of the results obtained for the right armpit, i.e., on which only alcohol is sprayed*): 3.62 g±0.3 g

Average of the Tests (average of the results obtained for the right armpit, i.e., on which alcohol solution and Jambu oleoresin solution is sprayed**): 2.68 g±0.6 g

Weight of Sweat Collected on Left Patches:

Average of the Control (see *): 1.73 g

Average of the Tests (see **): 2.41 g±0.3 g

Treated Armpit (Right Armpit)/Untreated Armpit (Left Armpit) Differential:

Average of the Control: 109%

Average of the Tests: 13.65%

Armpit Treated with Active Product (See **)/Armpit Treated with Placebo (See *) Differential:

Average of the Tests: −25.7%

### Example 3

Application either of 5 sprays (170 mg) of an 85% alcohol solution comprising 0.5% of supercritical CO₂ extract of Sichuan pepper containing 18% of sanshools and 1% of nerolidol only on the right armpit or of only 85% alcohol (control), and no application on the left armpit

After the effort, at t+1 h, the weight of sweat collected on the right patches is measured:

Average of the Control (average of the results obtained for the right armpit, i.e., on which only alcohol is sprayed *): 2.84 g±0.3 g

Average of the Tests (average of the results obtained for the right armpit, i.e., on which alcohol solution and Sichuan pepper extract solution is sprayed **): 2.25 g±0 g

Weight of sweat collected on the left Patches:

Average of the Control: 2.3 g±0.2 g

Average of the Tests: 2.31 g±0 g

Treated Armpit/Untreated Armpit Differential:

Average of the Control: 23.5%

Average of the Tests: −2.65%

Armpit Treated with Active Product/Armpit Treated with Placebo Differential

Average of the Tests: −20.75%

### Example 4

Application either of 5 sprays (170 mg) of an 85% alcohol solution comprising the following mixture on the right armpit.
0.25% of Jambu (Acmella oleacerae) Oleoresin CAS No. 90131-24-1, containing 32% of spilanthol (or 0.08% of pure spilanthol CAS No. 25394-57-4 (Robertet manufacture)), and
0.25% of CO₂ extract of Sichuan pepper (Zanthoxylum piperitum) CAS No. 102242-62-6, containing 18% of alpha and beta hydroxy sanshool, and
1% of nerolidol CAS No. 7212-44-4 or of only 85% alcohol (control), and no application on the left armpit

After the effort, at t+1 h, the weight of sweat collected on the right patches is measured:

Average of the Control (average of the results obtained for the right armpit, i.e., on which only alcohol is sprayed *): 3.79 g±0.3 g

Average of the Tests (average of the results obtained for the right armpit, i.e., on which alcohol solution, Jambu oleoresin solution and Sichuan pepper extract solution is sprayed **): 2.37 g±0.6 g

Weight of Sweat Collected on the Left Patches:

Average of the Control *: 3.54 g±0.4 g

Average of the Tests **: 2.56 g±0.5 g

Treated Armpit/Untreated Armpit Differential:

Control: 6%

Average of the Tests: −7.95%

Armpit Treated with Active Product/Armpit Treated with Placebo Differential

Average of the Tests: −32.9%

### Example 5

Aerosol composition according to the invention comprising:
Jambu Oleoresin (Acmella Oleacerae) CAS No. 90131-24-1, containing 30% of spilanthol: 0.3%
CO₂ extract of Sichuan pepper (Zanthoxylum piperitum) CAS No. 102242-62-6, containing 16% of alpha and beta hydroxy sanshool: 0.3%
Nerolidol CAS No. 7212-44-4: 1%,
Dicarylyl Carbonate: 2.6%
Cyclomethicone: 25.8%
AEROGAZ 2.5 (PROPANE-BUTANE-2.5 BARS): 70%

Methodology

The aerosol solution according to the invention was used on 14 adult volunteers aged 18 to 65, having any type of skin at the armpits and tending to sweat easily.

After a period of 10 days including the interruption of the application of any antiperspirant (wash-out) and the exclusive use of a deodorant without aluminum salts.
Day 1: application at the Center—5 sprays on the armpit to be treated according to a randomization scheme, no application on the other armpit.
At Day 2 and Day 3: applications at home—5 sprays on the armpit to be treated every day according to a randomization scheme,
Day 4: application at the Center—5 sprays on the armpit to be treated according to a randomization scheme.

Evaluation Criteria:
Evaluation by gravimetric method of the antiperspirant effectiveness 24 h following the last application, after a period of sweating in a heated room for 40 minutes preceded by an acclimation period of 20 minutes in the same conditions (collection of sweat during the thermal stimulation period and recording of the weighing of the buffer after the end of the thermal stimulation period).
Skin examination of the armpits: The possible presence of redness, skin dryness, little spots, tightness sensations, tingling sensations and itching was noted by the investigator or by the Clinical Assistant under the responsibility of the investigator.

Results of the Test

Number of volunteers: including 14 volunteers; 12 analyzed (2 test outputs).

Evaluation by gravimetric method of the antiperspirant efficacy—Armpits treated and control at the end of 24 hours following the last application (n=12)

| | Weight of collected | | |
|---|---|---|---|
| | sweat (in g) T24H | | Responding volunteers |
| | Treated | Control | (decrease ≥20% on the |
| | armpit | armpit | treated armpit) |
| Average | 0.616 | 0.725 | |
| Standard deviation | 0.593 | 0.610 | |
| Median | 0.430 | 0.465 | |
| Minimum | 0.216 | 0.212 | |
| Maximum | 2.362 | 2.225 | |

| | | |
|---|---|---|
| % change on the | −15.0% | 6/12 (50.0%) |
| average | | |

Conclusion: Under the retained experimental conditions, the product SPRAY ANTIPERSPIRANT according to the invention, showed an antiperspirant efficacy 24 hours after the end of a series of daily applications for 4 consecutive days at the armpit; the retained efficacy criterion being the following: obtaining a decrease of at least 20% of the amount of sweat collected on the treated armpit compared to the control armpit in at least 50% of the volunteers.

1. A method comprising applying at least one N-alkylamide as an antiperspirant.

2. A method comprising applying at least two different N-alkylamides as an antiperspirant.

3. The method according to claim 1, wherein the N-alkylamide is selected from the following alkylamides: spilanthol, trans-pellitorin; cis-pellitorin; 2Z,4Z- or 2Z,4E-decadienoic acid-N-isobutylamide; 2E,4E-decadienoic acid-N-([2S]-2-methylbutyl)amide; 2E,4E-decadienoic acid-N-([2R]-2-methylbutylamide); 2E,4Z-decadienoic acid-N-(2-methylbutyl) amide; achilleamide; sarmentine; 2E- or 3E-decenoic acid-N-isobutylamide; 3E-nonenoic acid-N-isobutylamide; homospilanthol; 2E,6Z,8E-decatrienoic acid-N-([2R]-2-methylbutyl)amide; 2E- or 2Z-decen-4-oic acid-N-isobutylamide; sanshools, or combinations thereof.

4. The method according to claim 1 wherein the N-alkylamide is the spilanthol and/or sanshools.

5. The method according to claim 4 wherein the source of spilanthol is an oleoresin extract of Acmella oleacerae or pure spilanthol and the source of sanshools is a supercritical CO₂ extract of Zanthoxylum piperitum.

6. The method comprising applying at least one N-alkylamide according to claim 1, in combination with a penetration enhancer.

7. The method comprising applying at least one N-alkylamide according to claim 1 wherein the composition is in the liquid form, in the solid form, or in the form of a gel.

8. A method for cosmetic treatment of the perspiration consisting in applying, locally, an effective amount of a composition comprising at least one N-alkylamide.

9. The method for cosmetic treatment according to claim 8 wherein a composition comprising at least two N-alkylamides is applied.
